(19) United States
(12) Patent Application Publication
(10) Pub. No.: US 2003/0149418 A1
(43) Pub. Date: Aug. 7, 2003

(54) Stand up diaper

(76) Inventor: Jill Katz, Pepper Pike, OH (US)

Correspondence Address: MCHALE & SLAVIN, 4440 PGA BLVD, SUITE 402, PALM BEACH GARDENS, FL, 33410

(21) Appl. No.: 10/072,447

(22) Filed: Feb. 7, 2002

(51) Int. Cl. A61F 13/15

(52) U.S. Cl. 604/385.25; 604/385.3; 604/386

(57) ABSTRACT

A diaper for quick changing in a confined space is preformed with a single leg opening and an opposite open side seam. The open side seam has cooperating fastener elements to fit and secure the diaper about an infant.

### FIELD OF THE INVENTION

This invention relates to the field of diapers for retaining accidental or uncontrolled bodily excretions. The diaper of this invention is directed to quick changing of an infant while the infant is standing or in the erect position.

### BACKGROUND OF THE INVENTION

The modern disposable diaper follows the design of the older cloth diaper. Currently, the diaper is generally rectangular in shape with a front panel and a back panel connected by a crotch portion defined by two opposing arcuate cut-outs approximately midway between the ends.

In use, the diaper is opened flat and the baby is laid upon the back panel of the diaper with the legs placed in the arcuate cut-outs. The front panel of the diaper is moved upwardly so that the side edges of the front panel contact the side edges of the back panel. There are fasteners on either the front panel or the back panel or both panels, usually adhesive strips, that are used to secure the diaper together about the baby's waist.

This type of diaper has at least two prerequisites, first, an adequate space for laying the baby down and second, a cooperative subject. If either or neither of these prerequisites is present, changing the subject's diaper can be extremely difficult for all concerned. Further, more time is necessary for such a diaper change. And the resulting fit of the diaper may be less than perfect because of misalignment of the fasteners.

Also, there comes a time in the development of the baby when standing is preferable to sitting or lying down. This period may last from age 6 months until the baby is potty trained.

What is needed in the art is a less complex diaper that can be applied quickly while the subject is standing up.

### DESCRIPTION OF THE PRIOR ART

U.S. Pat. No. 4,835,795 issued to Lonon discloses a sanitary brief for women with a crotch portion releasably fastened, at one end, to the body of the brief allowing use of the bathroom facilities without the necessity of removing the brief. In one embodiment, the brief has an open seam extending between the waistband and a leg opening held together by a releasable fastening, such as VELCRO. The openable side seam is for ease in washing and ironing.

Meisels, U.S. Pat. No. 5,103,501, discloses disposable underwear for traveling. The waistband of the man's brief and the woman's panty is discontinuous to allow donning of the garment without tearing. The waistband is provided with magnets for closing the seam.

U.S. Pat. No. 4,227,530 to Schatz discloses a disposable diaper with adhesive fasteners on both side seams. However, after the diaper is initially fitted to the wearer, one side fastener is capable of being opened without destroying the diaper. This allows for an inspection of the interior of the diaper or a later re-fitting of the waistband of the diaper.

### SUMMARY OF THE INVENTION

Accordingly, it is an objective of the instant invention to teach a diaper having one open side seam and one preformed leg opening. The open side seam has cooperating fasteners for securing the diaper about the waist of the infant forming a self supporting diaper.

It is a further objective of the instant invention to teach a diaper capable of a quick application by inserting an infant's leg through the pre-formed leg opening thereby securing the diaper about the baby. The open side seam is then fastened together adjusting the waistband and the other leg opening to fit the baby.

It is yet another objective of the instant invention to teach the diapering of an infant while the infant is standing or partially erect.

It is a still further objective of the invention teach the diaper of this invention having an liquid impermeable outer sheet, a liquid permeable inner cover sheet, an absorbent pad confined between the outer and inner sheets, and elastic margins forming a resilient waistband and leg openings.

Other objects and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

FIG. 1 shows a perspective of the diaper of this invention as applied to an infant; and

FIG. 2 shows a perspective of a modification of the diaper of FIG. 1.

### DETAILED DESCRIPTION OF THE INVENTION

The diaper 10 may be constructed of conventional disposable elements, such as a pervious inner layer 11 for contacting the body of the infant and an outer layer 12 that is impermeable to liquid for protecting the outer garments. An absorbent pad 13 is confined between the outer and inner layers. The pervious inner layer 11 may be nonwoven material that may be absorbent or may be hydrophobic. The hydrophobic material will act to wick urine from the baby's skin into the absorbent pad. In one embodiment, the inner layer may be slightly larger than the pad 13 and connected to the outer layer about the periphery of the pad.

The outer layer 12 is a thin film of a plastic polymer which is impermeable to liquids however, may have the ability to transmit gases for ventilation. The film is supple to allow for repeated creasing and stretching during the wearer's activities. In some cases the film may be re-enforced with woven or nonwoven yarns or threads embedded in the film. In one embodiment, the film may be slightly larger than the absorbent pad and connected to the inner layer about the periphery of the absorbent pad.

Usually, the inner and outer layers are substantially the same size and shape. The absorbent pad 13 usually is of smaller dimensions than the inner and outer layers. The pad may be a cellulosic batt of fibers 14. In some instances, the batt may incorporate super absorbent materials, such as starches or other polymers. The batt may be re-enforced by strands or yarns and may have patterns of compressed areas to direct the excretions into the interior of the pad. The pad may be secured to either the inner or outer layer, or both, or neither. However, the pad may be held in place by the bonding 15 of the inner and outer layers about the periphery of the pad. The bonding 15 may include substantially the entire area of contact between the inner and outer layers.

In another embodiment, the entire diaper may be made of materials, such as cloth and rubber sheeting, that are capable of repeated cleaning and use.

The front panel 16 is shaped to cover the entire surface of the wearer's abdomen from the waistband 19 to the crotch 20 and from side to side. The back panel 17 is likewise sized to cover the posterior of the wearer from the waistband 21 to the crotch and from side to side. The side juncture 18 of the front and back panels is an integral union.

The front and back panels are joined by a crotch portion 20 having arcuate edges 22 and 23. The arcuate edges 22 and 23 form the periphery of the leg openings 24 and 25. The crotch portion 20 may be formed as an integral portion of the front and back panel, as shown, or it may be attached to the front and back panel, respectively, by a permanent seam (not shown).

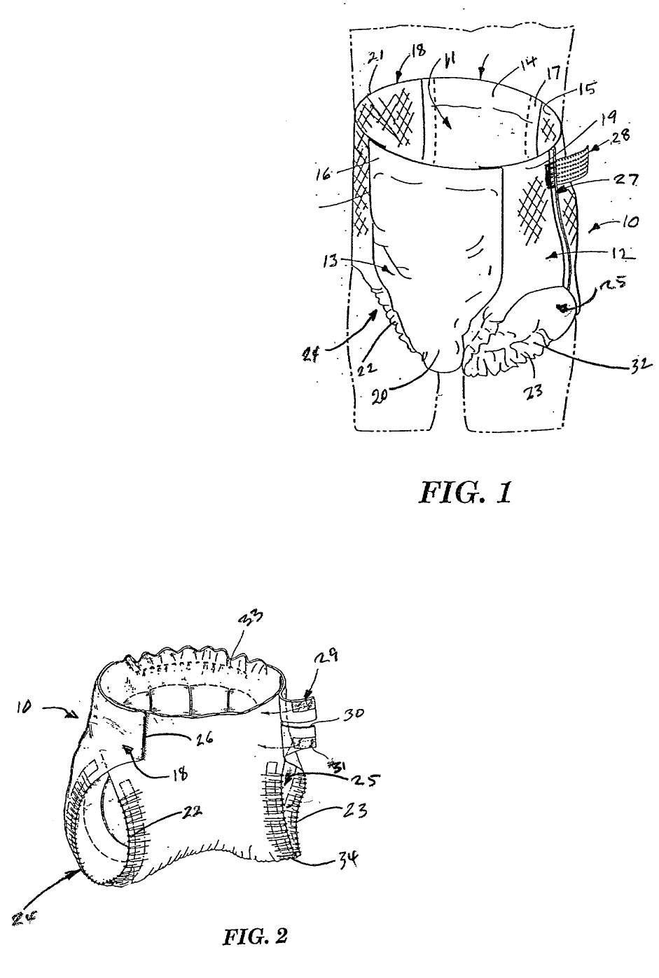

The side juncture 18, shown in FIGS. 1 and 2, along with the arcuate edge 22 of the crotch portion forms a permanent leg opening 24. While shown on the right side of the diaper, the permanent leg opening may be on either side of the diaper. The side juncture 18 may be formed by a continuous front and back panel, as-shown in FIG. 1, or by a permanent seam 26, shown in FIG. 2.

The opposite side 27 of the diaper is formed by the free ends of the front panel 16 and back panel 17. In FIG. 1, the front panel 16 has an attached fastener, such as a VELCRO tab 28, extending outwardly generally parallel with the waistband. The back panel 17 has a cooperating VELCRO patch, not shown, extending from the free end along the waistband. When these cooperating parts are brought together an adjustable and releasable diaper waistband is completed. Rather than VELCRO, the cooperating fasteners may be adhesive tabs, or hooks-and-loops, or buttons-and-button holes, or the like. The fasteners may have an elastic portion for greater flexibility in waistband adjustment.

In FIG. 2 the back panel 17 has an attached fastener, shown as an adhesive tab. The tab 29 has a split 30 which allows the fastener to more closely define the leg opening. The adhesive tab is brought forward and adhered to the impermeable outer layer in the area of the waistband. The lower portion 31 of the tab adheres to the front panel in the area of the leg opening. Of course, this fastener 29 may be of other constructions, as described above.

As shown in FIG. 1, the leg openings may be provided with elastic inserts 32 to provide a closer and more secure fit about the legs of the wearer. As shown in FIG. 2, the waist band and the leg openings are provided with elastic inserts 33 and 34. Of course, either the leg openings or the waistband or neither may be provided with elastic.

This diaper, with the permanent leg opening, facilitates changing the diaper because the insertion of the wearer's leg into the permanent leg opening secures the diaper to the infant. By merely bringing the other free ends of the diaper together and manipulating the single fastener, the diaper is fitted and adjusted to the individual. If the diaper change must be made in confined space or with a recalcitrant infant, the process may take place while the infant is standing or in a nearly standing position.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings.

I claim:

1. A diaper adapted for quick changing a wearer comprising a pre-formed body, said body having an integral front panel and a back panel for encircling the body of a wearer, said front panel having a free end, said back panel having a free end, said integral front panel and said back panel having an upper waistband portion, said body including a crotch panel connecting said integral front panel and said back panel, said crotch panel having opposed first and second lateral edges, said first lateral edge forming a first leg opening for encircling the leg of a wearer, said free end of said integral front panel and said free end of said back panel extending between said upper waistband portion and said second lateral edge of said crotch panel, said free end of said integral front panel and said back panel and said second lateral edge of said crotch panel adapted to form a second leg opening, said free end of said integral front and back panels carrying cooperating fastener elements for selectively connecting said integral front and back panels about the waist of a wearer.

2. A diaper of claim 1 wherein said pre-formed body further comprises an impermeable outer layer and an pervious inner liner for contacting the wearer.

3. A diaper of claim 1 wherein said waistband portion is resilient.

4. A diaper of claim 1 wherein said cooperating fastener elements are resilient.

5. A diaper of claim 1 wherein said lateral edges include an elastic material, said lateral edges adapted to form resilient leg seals.

6. A diaper of claim 5 wherein said opposite lateral edges are arcuate forming a reduced area of said narrow crotch panel midway between said front panel and said back panel.

7. A diaper of claim 5 wherein said waistband portion is resilient.

8. A diaper of claim 7 wherein said cooperating fastener elements are resilient.

9. A method of applying a diaper of claim 1 to a standing wearer including the steps of inserting one leg into said first leg opening, adjusting said diaper so that said crotch panel engages the standing wearer between the legs, wrapping said integral front and back panels about the torso of a wearer, bringing said each free end of said integral front and back panels into contact with each other, and securing said cooperating fastener elements together thereby adjusting said waistband portion about the wearer.
